(19) United States
(12) Patent Application Publication
(10) Pub. No.: US 2012/0015380 A1
(43) Pub. Date: Jan. 19, 2012

(54) ANTI-POLYETHYLENE GLYCOL ANTIBODY EXPRESSING CELL QUANTIFY ANY FREE POLYETHYLENE GLYCOL AND POLYETHYLENE GLYCOL-DERIVATIZED MOLECULES

(75) Inventors: Tian-Lu Cheng, Kaohsiung City (TW); Steven R. Roffler, Taipei City (TW); Kuo-Hsiang Chuang, Chiayi City (TW); Ssu-Jung Lu, Pingtung County (TW)

(73) Assignee: KAOHSIUNG MEDICAL UNIVERSITY, Kaohsiung City (TW)

(21) Appl. No.: 13/032,317

(22) Filed: Feb. 22, 2011

(30) Foreign Application Priority Data
Jul. 19, 2010 (TW) 099123699

(51) Int. Cl. G01N 33/98; C12N 5/071

(52) U.S. Cl. 435/7.92; 435/345

(57) ABSTRACT

In this invention, anti-PEG antibodies or anti-methoxyl-PEG (anti-CH₃O-PEG) antibodies were expressed on cell surface which can collocate with a biotinylated anti-PEG antibody (AGP4-Biotin) or biotinylated PEG (PEG-Biotin) to develop a cell-based sandwich ELISA or a cell-based competition ELISA, respectively. Both of these two methods could sensitively quantify free PEG and PEG-modified macromolecules (proteins, nanoparticles and liposomes) as sensitive as nano-gram level.

### FIELD OF THE INVENTION

This invention relates to construction of many kinds of cell lines presenting anti-polyethylene glycol backbone or anti-methoxyl-polyethylene glycol on their cell membranes, and developing a cell-based sandwich ELISA or a cell-based competition ELISA.

### BACKGROUND OF THE INVENTION

Polyethylene glycol (PEG) is a water-soluble, nontoxic, low immunogenic and biocompatible polymer that has been approved by the FDA for human usage by oral intake or intravenous and subcutaneous injection. Research shows that PEG has effects on preventing the colorectal cancer and healing the neuronal injury. Besides, the macromolecules (such as protein, nano drug and liposome) modified by PEG have the advantages on decreasing their immunogenicity, increasing half-life and biocompatibility. However, there is still no convenient way to effectively quantify PEG or PEG-modified (PEGylated) molecules in vivo.

Current methods used for quantifying PEG, for example: (a) Using traditional ELISA, as known as Sandwich Enzyme-Linked Immunosorbent Assay, to directly recognize protein, but the detecting process is hard to be achieved and the results are not precise due to the PEG used to modify proteins often cover the epitope for antibodies to recognize. Moreover, using ascites to produce antibodies has been prohibited by European and the US government, and this results the price of products related to ELISA to raise in the future. (b) A colorimetry which use barium-iodide or Fe(CN)3 and PEGylated protein to form a compound need to get rid of other proteins prior to detect, and sensitivity of this method is relatively low, only reach about 1-5 μg/ml ° (C.) To detect PEG by isotope labeling PEGylated molecule. Although the sensitivity for this method is good, this method can not be used in every PEGylated molecule. Besides, due to the production of radioactive waste, this method can only be performed by specific operators in specific lab, causing inconvenience and leading to unpopularity. Therefore, developing a method which is simple, sensitive, cheap, and able to detect the pharmacokinetics of any PEGylated molecules in vivo can solve those problems caused by traditional methods, just like inconvenience, low sensitivity, and expensive. Also, this new method can be used in any labs, helping labs to evaluate the pharmacokinetics of PEGylated molecules in vivo or in vitro. This method will play an important role in developing PEGylated drug in the future.

### SUMMARY OF THE INVENTION

This invention relates to stably express a functional anti-PEG antibody on the surface of BALB 3T3 cells (3T3/αPEG cells) to develop a sandwich ELISA or a competitive ELISA for PEG quantification.

### BRIEF DESCRIPTION OF THE DRAWINGS

FIG. 1. 3T3/AGP3, 3T3/E11, 3T3/3-3 and 3T3/6-3 cells can recognize the repeat sequence of PEG (—O—CH2—CH2—)n, while 3T3/15-2 cells can recognize the methoxyl-PEG (CH3O-PEG). Therefore, any free PEG molecules and PEGylated macromolecules (e.g. liposome, protein, nano particle, drug, etc.) can be recognized by such kind of cells.

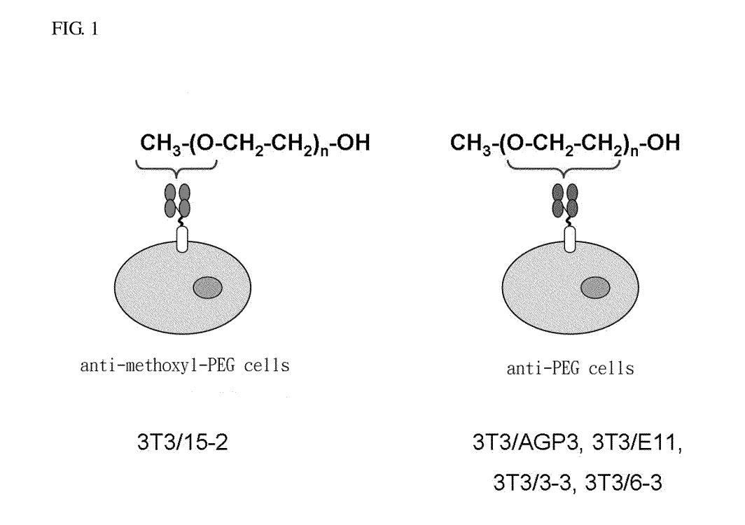

FIG. 2. (A) Detect the expression level of membrane protein antibodies presented on the 3T3/AGP3 cells with HA (hemagglutinin) antibody. Test the function of AGP3 cell membrane antibodies to recognize PEG with PEG-Q-dot. (B) Detect the expression level of membrane protein antibodies presented on the 3T3/15-2 cells with HA (hemagglutinin) antibody. Test the function of 15-2 cell membrane antibodies to recognize CH3O-PEG with PEG-Q-dot or CH3O-PEG-BSA-FITC. The result shows that both AGP3 and 15-2 cell membrane antibodies can highly express on the surface of 3T3 cells; 3T3/AGP3 cells can recognize the backbone of PEG, while 3T3/15-2 cells can only recognize the methoxyl-PEG (CH3O-PEG) but not PEG-Q-dot (PEG without methoxyl).

FIG. 3. The sandwich ELISA based on 3T3/AGP3 platform can sensitively detect (A) free PEG molecules up to 100 ng/ml, (B) PEG-Interferon 2α up to 10 ng/ml, (C) Lipo-Dox up to 10 ng/ml, and (D) PEG-Q-dot up to 0.1 nM; 3T3/DNS is the negative control.

FIG. 4. The sandwich ELISA based on 3T3/15-2 platform is more sensitive in detecting free methoxyl-PEG (CH3O end) molecule than 3T3/AGP3; (A) 5 KDa, (B) 2 KDa; 3T3/DNS is the negative control.

FIG. 5. The competitive ELISA based on 3T3/AGP3 platform. (A)Compete different concentrations and different molecular weight (MW: 2, 5, 10 and 20 KDa) of free PEG molecules with biotinylated PEG (125 ng/ml). The light absorbance shows linear decrease with the raise of the concentration of free PEG molecules when the concentration of free PEG molecules (2, 5, 10 and 20 KDa) is up to 58.6, 14.6, 3.7 and 3.7 ng/ml, respectively. It proves that the competitive ELISA effectively quantifies free PEG molecules and its sensitivity is up to ng/ml scale. (B) Prepare PEG-Biotin and free PEG molecules (MW: 5 KDa) in the solution with different serum concentrations (2.5%-40%) and perform competitive ELISA. The result shows that even though the percentage of serum is up to 40%, there is no influence on the ability of competitive ELISA to quantify the amount of free PEG molecules.

FIG. 6. The expression level and function of E11, 3-3 and 6-3 cell membrane antibodies. Test the protein expression level of anti-PEG membrane antibodies on the 3T3 cells with anti-HA antibody (solid line) and test the function of anti-PEG membrane antibody to recognize PEG with PEG-Q-dot (dotted line). (A) 3T3/E11, (B) 3T3/3-3, (C) 3T3/6-3. The result shows that those three cell lines are able to bind PEG effectively. The result also shows that sandwich ELISA and competitive ELISA can use those three cell lines as a platform to detect free PEG molecules and PEGylated macromolecules.

### DETAILED DESCRIPTION OF THE INVENTION

In this invention, anti-PEG antibodies or anti-methoxyl-PEG (anti-CH3O-PEG) antibodies were expressed on cell surface which can collocate with a biotinylated anti-PEG antibody (AGP4-Biotin) or biotinylated PEG (PEG-Biotin) to develop a cell-based sandwich ELISA or a cell-based competition ELISA, respectively. Both of these two methods could sensitively quantify free PEG and PEGylated macromolecules (proteins, nanoparticles and liposomes) as sensitive as nano-gram level. Compared to the traditional ELISA which directly recognizes antibody protein, the cell-based ELISA has advantages, such as: (1) the high growing rate of 3T3 cells, and only a little of serum (or even no serum) is needed for culturing the cells to obtain great amount of cells which present anti-PEG antibody on cell membrane. (2) the antibody presenting on the cell membrane is in a consistent direction, and the antigen binding domains are heading toward outside, which can effectively improve the sensitivity of detecting PEG (3) the size of the anti-PEG antibody presenting 3T3 cell is similar to micro-particle, which can effectively increases the surface for reaction to improve the sensitivity of detecting PEG.

Present invention relates to a detection kit for detecting polyethylene glycol (PEG), which comprises an anti-PEG presenting cell which presents the anti-PEG antibody on its membrane, wherein the antibody is presented in a consistent direction on the membrane. Present invention also relates to a detection method for detecting PEG There are two ways to detect PEG: The first way is fixing a cell which presents the anti-PEG antibody on its membrane on a steady holder, and then sequentially adding analyte PEG or PEGylated molecules, chemical modified anti-PEG antibody, and color reagent. The PEG concentration can be quantified by observing color intensity after washing out the extra remaining color reagent. The second way is fixing a cell which presents the anti-PEG antibody on its membrane on a steady holder, and then adding equal volumns of biotinylated PEG and analyte PEG or PEGylated molecules at the same time. Finally, adding color reagent and washing out the remaining color reagent and quantifying the concentration of PEG by the color intensity.

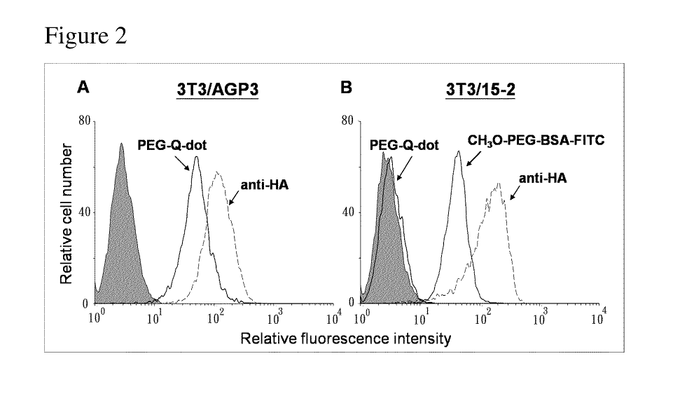

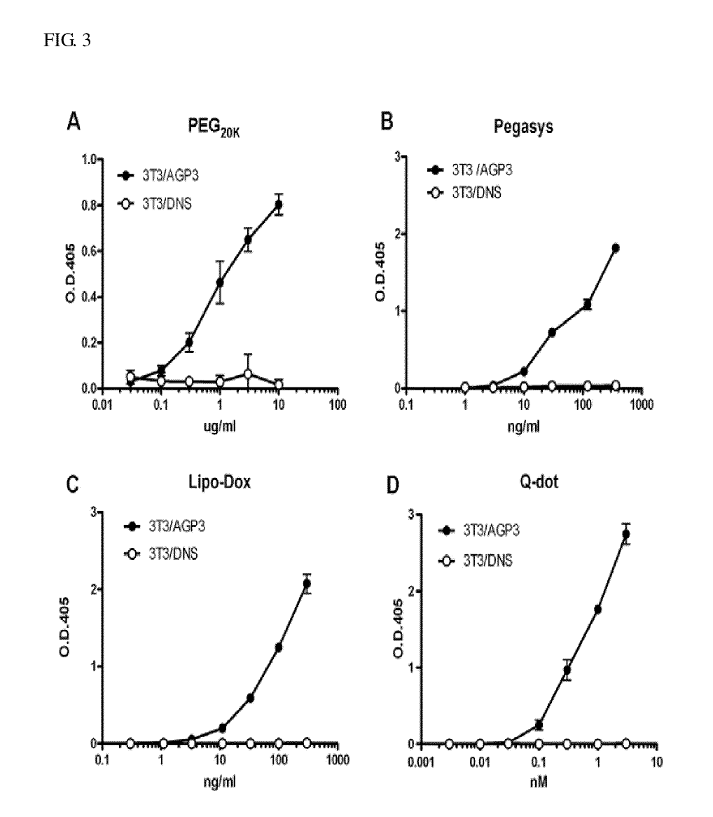

This invention presents all kinds of anti-PEG antibodies (AGP3/IgM [symbol] E11/IgG1 [symbol] 3-₃/IgG1 [symbol] 6-3/IgG1) or an anti-methoxyl-PEG antibody (15-₂/IgG2b) to the cell membrane of mammalian cells, such as mouse embryonic fibroblast cells (3T3) to construct a cell line able to quantify all kinds of PEG and PEGylated molecules. In one embodiment of the present invention: (1) the light chain (VL-CK) and heavy chain (VH-CH1) of Fab antibody sequence of the AGP3 antibody or the 15-2 antibody were connected with the foot-and-mouth virus 2A peptide, and further combined with the immunoglobulin C2-type extracellular-transmembrane-cytosolic domains of mouse B7 antigen. (2) Afterward, integrated (1) to the virus vector pLNCX to make the pLNCX-AGP3 Fab-B7 and pLNCX-15-2 Fab-B7 plasmid, respectively. (3) GP2-293 cells were infected with said plasmid and pVSVG vector (a retro virus which led to defects on reproduction for GP2-293) by lipofectamine 2000. 48 hours later, the medium of GP2-293 had retro virus particles which contain AGP3 Fab-B7 gene and 15-2 Fab-B7 gene, respectively. (4) 3T3 cell line was infected with said retro virus particles and infected cells were selected with antibiotics G418 sulfate. (5) At last, those high expression cells were collected by flow cytometry, and then the 3T3/AGP3 cells and 3T3/15-2 cells were obtained. (FIGS. 2A and 2B). A sandwich ELISA can be established by using 3T3/AGP3 cells or 3T3/15-2 cells with biotinylated anti-PEG antibody (AGP4-Biotin). In one embodiment of the present invention, 3T3/AGP3 cells were seeded into a 96 well plate before the PEG or PEGylated macromolecules was added, and then a biotinylated anti-PEG antibody was added as a detecting antibody. Finally, streptavidin-HRP was added. After adequate washing, the concentration of PEG or PEGylated molecules can be known by observing the color intensity of the matrix coated on the 96 well plate. The 3T3/AGP3 cell-based sandwich ELISA can effectively quantify free PEG molecules having molecule weight 20KD (sensitivity up to 100ng/m1) (FIG. 3A), PEGylated Interferon (PEG-Interferon 2α, Pegasy) (sensitivity up to 10 ng/ml) (FIG. 3B), PEGylated liposomal drug (Lipo-Dox) (sensitivity up to 10 ng/ml) (FIG. 3C), and PEGylated fluorescence nanoparticle (PEG-Quantum dots, PEG-Q-dot) (sensitivity up to 0.1 nM) (FIG. 3D). More importantly, comparing to 3T3/AGP3 cells, the 3T3/15-2 cell-based sandwich ELISA can quantify the free PEG molecules which have methoxyl group more sensitive.

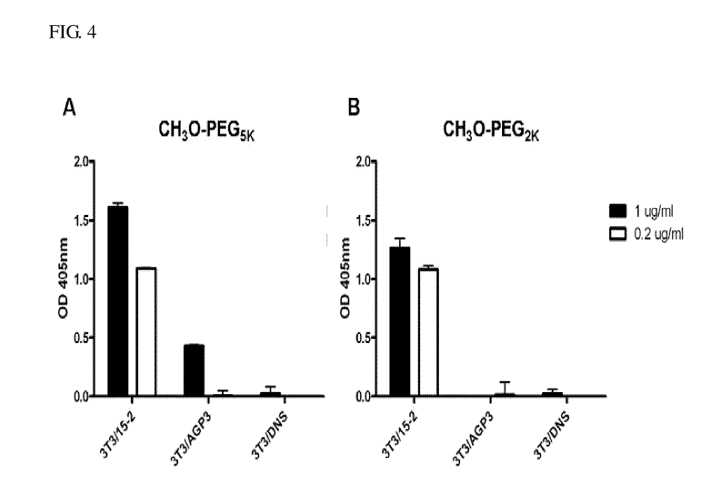

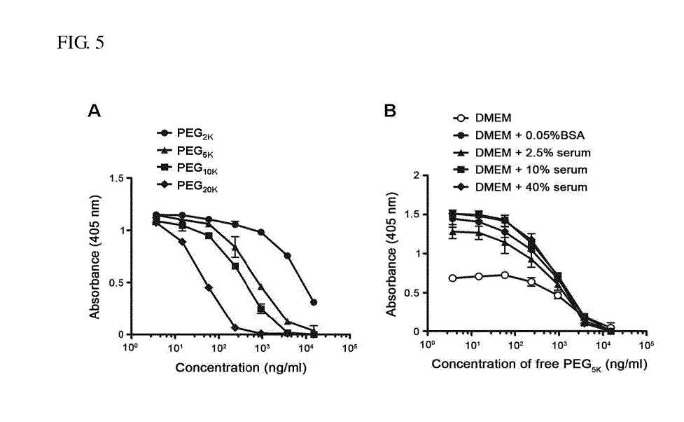

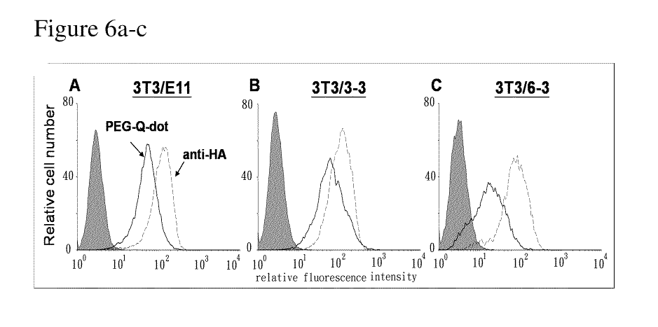

The present invention finds that, comparing to 3T3/AGP3 cells, the sandwich ELISA based on 3T3/15-2 cells can more sensitively quantify free methoxyl-PEG For the methoxyl-PEG with molecular weight 5,000 and 2000, the detection sensitivity of 3T3/15-2 cell-based sandwich ELISA is respectively 3.7 times and 100 times higher than 3T3/AGP3 cell-based sandwich ELISA do (FIG. 4). Besides, for quantification of non-methoxyl PEG molecules, the present invention collocates 3T3/AGP3 cells which detects PEG back-bone with biotinylated PEG to develop a competitive ELISA. Competitive ELISA can be applied by mixing equal volumns of biotinylated PEG with the PEG sample, or PEGylated molecules which has different molecular weights (MW: 2K-20 KDa). The competitive ELISA can sensitively quantify free PEG molecules with MW 2 KDa, 5 KDa, 10 KDa and 20 KDa. The sensitivity is up to 58.6, 14.6, 3.7, and 3.7 ng/ml, respectively (FIG. 5A), and is not influenced by serum (FIG. 5B). Present invention also construct other cell lines which present anti-PEG antibodies on their cell membranes, such as 3T3/E11 cells, 3T3/3-3 cells and 3T3/6-3 cells (FIG. 6). These cell lines can be applied on a sandwich ELISA or a competitive ELISA as well.

### EXAMPLE

The examples below are non-limiting and are merely representative of various aspects and features of the present invention.

### Example 1

Anti-PEG cells were used as a platform to establish a sandwich ELISA to quantify free PEG molecules and PEGylated molecules. 3T3/AGP3 cells were seeded into a 96 wells plate before the PEG or PEGylated molecules were added, and then a biotinylated anti-PEG antibody (AGP4-Biotin) was used as a detecting antibody. Finally, color reagent, streptavidin-HRP, was added into the 96 wells plate. After adequate washing, the concentration of PEG or PEGylated molecules were known by observing the color intensity of the matrix coated on the 96 wells plate. With existence of serum, this method can quantify PEG molecules and PEGylated macromolecules (such as protein, fluorescent-nanoparticle and liposome) with PEG molecular weight 2,000, respectively, and the sensitivity is up to 100 ng/ml (5 nM), 10 ng/ml, 0.1 nM and 10 ng/ml, respectively. Therefore, this method can sensitively quantify all kinds of PEGylated molecules.

### Example 2

Anti-methoxyl-PEG cells were used as a platform to establish a sandwich ELISA to quantify all kinds of free methoxyl-PEG molecules. 3T3/15-2 cells were seeded into a 96 wells plate. After that, the methoxyl-PEG was added as an analyte, and then a biotinylated anti-PEG antibody (AGP4-Biotin) was used as a detecting antibody. Finally, streptavidin-HRP was added into the 96 wells plate. After adequate washing, the concentration of methoxyl-PEG was known by observing the color intensity of the matrix coated on the 96 wells plate. With existence of serum, this method can quantify all kinds of free methoxyl-PEG molecules (CH3O-PEG2K and CH3O-PEG5K), and the sensitivity was better than using 3T3/AGP3 platform. Therefore, the sandwich ELISA method using 3T3/15-2 platform can sensitively quantify all kinds of methoxyl-PEG molecules.

### Example 3

Anti-PEG cells were used as a platform to establish a competitive ELISA to quantify free PEG molecules. 3T3/AGP cells were seeded into a 96 wells plate, and a fixed amount of the biotinylated-PEG molecule (PEG-Biotin) was mixed with the analyte (free PEG molecule) by equal volume (1:1) to form a mixture, and then the mixture was added into the 96 wells plate. Finally, streptavidin-HRP was added into the 96 wells plate. After adequate washing, the concentration of free PEG molecule can be known by observing the color intensity of the matrix coated on the 96 wells plate. With existence of 40% serum, this method can quantify PEG molecules with molecular weight 20,000 to 2,000 (PEG20K, PEG10K, PEG5K and PEG2K), and the sensitivity is up to 3.7 ng/ml, 3.7 ng/ml, 14.6 ng/ml and 58.6 ng/ml, respectively. Therefore, this platform can sensitively quantify many kinds of free PEG molecules.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The antibodies, processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A kit for detecting polyethylene glycol (PEG), comprising:
an anti-PEG antibody presenting cell which presents anti-PEG antibody on a membrane surface of the cell.

2. The polyethylene glycol (PEG) detecting kit of claim 1, wherein the cell is mammalian cell.

3. The polyethylene glycol (PEG) detecting kit of claim 2, wherein the mammalian cell is 3T3 cell.

4. The polyethylene glycol (PEG) detecting kit of claim 1, wherein the anti-PEG antibody detect the backbone of PEG.

5. The polyethylene glycol (PEG) detecting kit of claim 4, wherein the anti-PEG antibody is selected from the group consisting of AGP3, Ell, 3-3 and 6-3.

6. The polyethylene glycol (PEG) detecting kit of claim 1, which further comprises a biotinylated anti-PEG antibody.

7. The polyethylene glycol (PEG) detecting kit of claim 6, wherein the biotinylated anti-PEG antibody is AGP4-biotin.

8. The polyethylene glycol (PEG) detecting kit of claim 1, wherein the antibody is capable of detecting the methoxyl end of methoxyl-PEG.

9. The polyethylene glycol (PEG) detecting kit of claim 8, wherein the antibody is 15-2 antibody.

10. The polyethylene glycol (PEG) detecting kit of claim 1, which further comprises a biotinylated PEG.

11. The polyethylene glycol (PEG) detecting kit of claim 10, wherein the biotinylated PEG is PEG-biotin.

12. The polyethylene glycol (PEG) detecting kit of claim 1, which further comprise a color reagent.

13. The polyethylene glycol (PEG) detecting kit of claim 12, wherein the color reagent is streptavidin-HRP.

14. A method for detecting PEG, comprising:
fixing a cell which presents anti-PEG antibody on its membrane on a steady holder;
adding analyte PEG or PEGylated molecules;
adding chemical modified anti-PEG antibody;
adding color reagent; and
washing out the extra remaining color reagent and quantifying the concentration of PEG by the color intensity.

15. The method of claim 14, wherein the cell is 3T3 cell.

16. The method of claim 14, wherein the anti-PEG antibody is selected from the group consisting of AGP3, E11, 3-3, 6-3 and 15-2.

17. The method of claim 16, wherein the 15-2 antibody is used to detect the methoxyl-PEG.

18. The method of claim 14, wherein the chemical modified anti-PEG antibody is biotinylated anti-PEG antibody.

19. The method of claim 14, wherein the chemical modified anti-PEG antibody is AGP4-biotin.

20. The method of claim 14, wherein the color reagent is streptavidin-HRP.

21. A method of detecting PEG, comprising:
fixing a cell which presents anti-PEG antibody on its membrane on a steady holder;
adding equal volumes of biotinylated PEG and analyte PEG or PEGylated molecule at the same time;
adding color reagent; and
washing out the extra remaining color agent and quantifying the concentration of PEG by the color intensity.

22. The detecting method of claim 21, wherein the cell is 3T3 cell.

23. The detecting method of claim 21, wherein the anti-PEG antibody is selected from the group consisting of AGP3, E11, 3-3 and 6-3.

24. The detecting method of claim 21, wherein the biotinylated PEG is PEG-biotin.

25. The detecting method of claim 21, wherein the color reagent is streptavidin-HRP.


SEQUENCE LISTING (10 sequences)

SEQ ID NO: 1 - DNA, 348 bp, Artificial
  VH domain of AGP3 antibody
gaagtgcagctggtggagtctgggggaggcttagtgaagcctggagggtccctgaaactctcctgtgcagcctctggattcactttcagtgactattacatgtattgggttcgccagactccggaaaagaggctggagtgggtcgcaaccattagtgatgatggtacttacacctactatccacacagtgtgaaggggcgattcaccatctccagagacagtgccaagaacaacctgtacctgcaattgagcagtctgaagtctgaggacacagccatgtattactgtgcaagaaatgatgctaggggggactactggggtcaaggaacctcagtcaccgtctcctca

SEQ ID NO: 2 - DNA, 336 bp, Artificial
  VL domain of AGP3 antibody
gatattgtgttgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaactattcacgttcggctcggggacaaagttggaaataaaacgg

SEQ ID NO: 3 - DNA, 357 bp, Artificial
  VH domain of E11 antibody
caggtgcagctgcaggagtccggagctgaactggcaagacctggggcctcagtgatgatgtcctgcaaggcttctggctacacctttactacctacacgatgaactgggtaaaacagaggcctggacagggtctggaatggattggatacattattcctagcagtgggtatgttgactacaatcagaagttcaagggaaagaccatattgactacagacaaatcctccagcacagcctacatgcaactgagcagcctgacatctgaggactctgcggtctattactgtgtaagatcgcttgatggttatttctggtttgcttactggggccaagggactctggtcactgtctctgca

SEQ ID NO: 4 - DNA, 339 bp, Artificial
  VL domain of E11 antibody
gatgttttgatgacccaaagtccactctccctgcctgtcagtcttggagatcacgcgtccatctcttgcagatcaagtaagagcattgtacacagtaatggaaacacctatttggaatggttcctgcagaaaccaggccagtctccaaaactcctgatctacaaagtttccaagcgaatgtctggggtcccagacaggttcagtggcagtggatcagggacagatttcacactcaagatccgcagagtggaggctgaggatctgggagtttattactgctctcaaggttcacatgttcctcccacgttcggaggggggaccaaactggaaataaaacgg

SEQ ID NO: 5 - DNA, 354 bp, Artificial
  VH domain of 3-3 antibody
gaagtcaagctggaggagtctggaggaggcttggtgcaacctggaggatccatgaaactctcttgtgctgcctctggattcatttttagtgacgcctggatggactgggtccgccagtctccagagagggggcttgagtgggttgctgaaattagaagcaaagctaatggtcttgcaccatactatgctgagtctgtgaaagggaggttcaccatctcaagagatgattccaaaagtagtgtctacctgcaaatgaacaacttaagatctgaagacactggcatttattactgtacctctactttgtattactttgactactggggccaaggcaccacgctcacagtctcctca

SEQ ID NO: 6 - DNA, 327 bp, Artificial
  VL domain of 3-3 antibody
cagattgtgctcacacagtctccagcaatcatgtctgcatttcctggggagagggtcaccttgacctgcagtgccagctcgagtgtgaggtccagctacttgtgctggtaccagcagaagccaggatcctcccccaaactctggatttatagcacttccaacctggcttctggagtccctgctcgcttcagtggcagtgggtctgggacctcttactctctcacaatcagcagcatggaggctgaagatgctgcctcttatttctgccatcagtggagtagttacccacggacgttcggtggaggcaccaagctggaaatcaaacgg

SEQ ID NO: 7 - DNA, 342 bp, Artificial
  VH domain of 6-3 antibody
cagatccagttggtgcagtctggacctgagctgaagaagcctggagagacagtcaagatatcctgcaaggcttctgggtatactttcaaaaactatggaatgaactgggtgaagcaggctccaggaaagggtttaaagtggatgggctggataaacacctacactggacagccaatatatgctaatgacttcaagggacggtttgccttctctttggaaacctctgccagcactgcctatttgcagatcaacaacctcaaaaatgaggacacggctacatatttctgtgcaagagactggggaccttactggggccaagggactctggtcattgtctctgca

SEQ ID NO: 8 - DNA, 339 bp, Artificial
  VL domain of 6-3 antibody
aacattatgatgacccagtctccatcatctctggctgtttctgcaggagaaaaggtcactgtgaactgtaagtccagtcaaagtgttttatacagttcaaatcagatgaactacttggcctggtaccagcagaaaccaggacagtctcctaaactgctgatctactgggcatccactagggaatctggtgtccctgatcgcttcacaggcagtggatctgggacagattttactcttaccatcagcagtgtacaaactgaagacctggcagtttattactgtcttcaatacctctcctcgtggacgttcggtggaggcaccaagctggaaatcaaacgg

SEQ ID NO: 9 - DNA, 342 bp, Artificial
  VH domain of 15-2 antibody
gaggttaagctggaggagtctggaggaggcttggtgcaacctggaggatccatgaaactctcctgtgttgcctctggattcactttcagtaactactggatgaactgggtccgccagtctccagagaaggggcttgagtgggttactgaaattagatcgaaatctaataattatgcgacacattatgcggagtctgtgaaagggaggttcaccatctcaagagatgattccaaaggtagtgtctacctgcaaatgaacaacttaagagctgaagacactggcatttattactgttccaacagatactactggggccaaggtaccctggtcactgtctctgca

SEQ ID NO: 10 - DNA, 324 bp, Artificial
  VL domain of 15-2 antibody
gacattgtgatgacacagtctcacaaattcatgtccacatcagtacgagacagggtcaccatcacctgcaaggccagtcaggatgtaaatacttctgtagcctggtatcaacagaaaccagggcaatctcctaagctggtgatttattgggcatccacccggcacactggagtccctgatcgcttcacaggcagtggatctgggacagatttcactctcaccattagcaatgttcagtctgaagacttggcagattatttctgtctgcaatatatcaactatccgtacacgttcggaggggggaccaagctggaaataaaacgg